(19) United States
(12) Patent Application Publication
(10) Pub. No.: US 2002/0155999 A1
(43) Pub. Date: Oct. 24, 2002

(54) Method of using a porphyrin-like molecule conjugated with an anti-cancer drug for the treatment of cancer

(76) Inventor: In Suk Han, Salt Lake City, UT (US)

Correspondence Address: MALLINCKRODT & MALLINCKRODT, SUITE 510, 10 EXCHANGE PLACE, SALT LAKE CITY, UT, 84111, US

(21) Appl. No.: 10/104,896

(22) Filed: Mar. 22, 2002

Related U.S. Application Data
(63) Continuation of application No. 09/303,217, filed on Apr. 30, 1999, now abandoned.
(60) Provisional application No. 60/083,929, filed on Apr. 30, 1998.

(51) Int. Cl. A61K 38/16; C07K 9/00; C07H 17/00; A61K 31/7052; A61K 31/555

(52) U.S. Cl. 514/8; 514/34; 514/185; 514/27; 514/410; 514/6; 514/176; 530/322; 536/8; 536/17.4; 540/145; 540/465

(57) ABSTRACT

An anti-cancer substance has a porphyrin-like molecule conjugated to an anti-cancer drug. In one embodiment, the porphyrin-like molecule is conjugated directly to an anti-cancer drug. In a second embodiment, the porphyrin-like molecule is conjugated to a first end of a peptide chain, while a second end of the peptide chain is conjugated to the anti-cancer drug. The peptide chain is designed to be cleaved under physiological conditions surrounding the tumor. In the preferred embodiment, the peptide chain functions as a protease inhibitor once it has been cleaved.

### RELATED APPLICATION

This application is a continuation of application Ser. No. 09/303,217 filed Apr. 30, 1999.

### BACKGROUND OF THE INVENTION

This application for a utility patent follows a previously filed provisional patent having the Serial No. 60/083,929 and a filing date of Apr. 30, 1998.

1. Field of the Invention

This invention relates generally to anti-cancer drugs, and more particularly to an anti-cancer drug conjugated to a porphyrin-like molecule to maximize therapeutic effects and minimize toxicity to non-tumor cells.

2. Description of Related Art

I. Porphyrin Molecules Tend to Become Localized in Rapidly Growing Neoplastic Tissues Such as Tumors

Porphyrin and porphyrin-like molecules are found in animals and plants. Ring structure of the porphyrin nucleus is biosynthesized and incorporated with a metal ion through a series of complex enzymatic reactions. Heme, a porphyrin member, is a prosthetic group which acts as a cofactor in heme proteins such as hemoglobin and the cytochromes. Heme consists of a planar tetrapyrrole ring system with a chelated iron ion at the center. Porphyrin-like molecules such as vitamin B12 and chlorophyll contain metal ions such as cobalt and magnesium in the porphyrin nucleus. Heme has two carboxyl groups and hydrophobic methyl and vinyl groups. These ring-like molecules call absorb light and transfer excitations to other forms of chemical and physical energy. The porphyrin heave been shown unique biological properties such as highly selective localization in rapidly growing neoplastic tissues such as tumors. These properties have been demonstrated in U.S. Pat. Nos. 5,733,903, 5,622,685, 5,162,519, 5,162,231, 4,992,257, and 4,783,529. See also Auber, H., and Banger, H. Krebsforsch 53:65-68 1942, Figge, F H., et al. Proc. Soc. Exp. Biol. Med. 68:640-641 1948.

II. Cancer Cells Produce Unusually High Concentrations of Certains Enzymes and Growth Factors

Cancer is caused by mutations of several genes in the cell. Any cellular tissue can become cancerous if the DNA of the cell is damaged. Such damage to cellular DNA can be caused by a variety of environmental conditions, including chemicals, radiation, and viruses. The mutated genes change the pattern of gene expression, cell growth pattern, and cell mitosis resulting in uncontrolled growth and proliferation of the cancerous cells. Cancer cells are defined by two hereditary tendencies: they and their progeny (1) reproduce in an uncontrolled fashion into a relentlessly growing mass of abnormal cells, and they (2) metastasize and spread throughout the body.

To actuate these abnormal behaviors, the cancerous cells must produce abnormal levels of various enzymes and growth factors. One specific abnormality involves the unusually large demand for nutrients required by cancerous cells. The growth of a solid tumor is limited by the diffusion of nutrients from its surroundings. To enlarge further, a tumor must induce angiogenesis, a process of capillary network formation, to supply nutrients inside of cancer cells. In order to form a capillary in the tumor, cancer cells secret growth factors such as vascular endothelial growth factors and fibroblast growth factors to induce angiogenesis from endothelial cells. I he endothelial cells respond to the signals, and move toward the source of the signal. In the process of breaching the basal lamina that surrounds an existing blood vessel, the endothelial cells produce proteases, which enable them to digest their way through the basal lamina of the parent capillary or venule. Thus, angiogenesis is a critical factor for the growth of tumor that requires a blood supply; and angiogenesis produces unusually high concentrations of certain types of proteases.

Cancer cells also spread, or metastasize, through the blood stream or lymphatic vessels to invade and colonize other normal tissues to form numerous secondary tumors. To metastasize, cancer cells must cross the basal laminae. The basal laminae is made of various proteins, including: type IV collagen, laminin, entactin, and perlecan. To digest vascular basal laminae and/or extracelluar matrix, extracelluar proteolytic enzymes are locally secreted by cancer cells. Most of these proteases are metalloproteases such as the collagenases and serine proteases such as plasmin and urokinase-type plasminogen activator (U-PA). Collagenases cleave highly specific positions of proteins. However, U-PA and plasmin cleave a variety of proteins such as fibrin, fibronectin, and laminin with a broad specificity.

As described above, it is known to those skilled in the art that porphyrins and porphyrin-like molecules (“porphyrin-like molecules”) have been utilized as photosensitizing agents for radiation therapy and diagnosis of cancers. Porphyrin-like molecules are particularly useful as photosensitizers because these molecules exhibit the preferred accumulation within tumors; and the porphyrin-like molecules tend to absorb X-ray energy to produce cytotoxic free radicals. Also as described above, it is known to those skilled in the art that tumors tend to produce higher levels of certain enzymes and growth factors in the process of growing and metastasizing.

The prior art teaches the use of porphyrin derivatives as photosensitizing agent. However, the prior art does not teach the conjugation of a porphyrin-like molecule with an anti-cancer drug to provide a particularly potent anti-cancer substance. The present invention fulfills these needs and provides further related advantages as described in the following summary.

### SUMMARY OF THE INVENTION

The present invention teaches certain benefits in construction and use which give rise to the objectives described below.

The present invention provides a method of targeted delivery of an anti-cancer drug and/or protease inhibitors to tumors. The invention utilizes a novel anti-cancer substance that include a porphyrin-like molecule conjugated to an anti-cancer drug. In one embodiment, the porphyrin-like molecule is conjugated directly to an anti-cancer drug. In a second embodiment, the porphyrin-like molecule is conjugated to a first end of a peptide chain, while a second end of the peptide chain is conjugated to the anti-cancer drug. The peptide chain is designed to be cleaved under physiological conditions surrounding the tumor. In the preferred embodiment, the peptide chain functions as a protease inhibitor once it has been cleaved.

A primary objective of the present invention is to provide an anti-cancer substance having advantages not taught by the prior art.

Another objective is to provide an anti-cancer substance that is capable of targeting an anti-cancer drug directly to the tumor.

A further objective is to provide an anti-cancer substance that releases an anti-cancer drug under the physiological conditions that surround the tumor.

Other features and advantages of the present invention will become apparent from the following more detailed description, taken in conjunction with the accompanying drawings, which illustrate, by way of example, the principles of the invention.

### BRIEF DESCRIPTION OF THE DRAWING

The accompanying drawings illustrate the present invention. In such drawings:

FIG. 1 is a flow diagram showing the synthesis of one embodiment of the present invention.

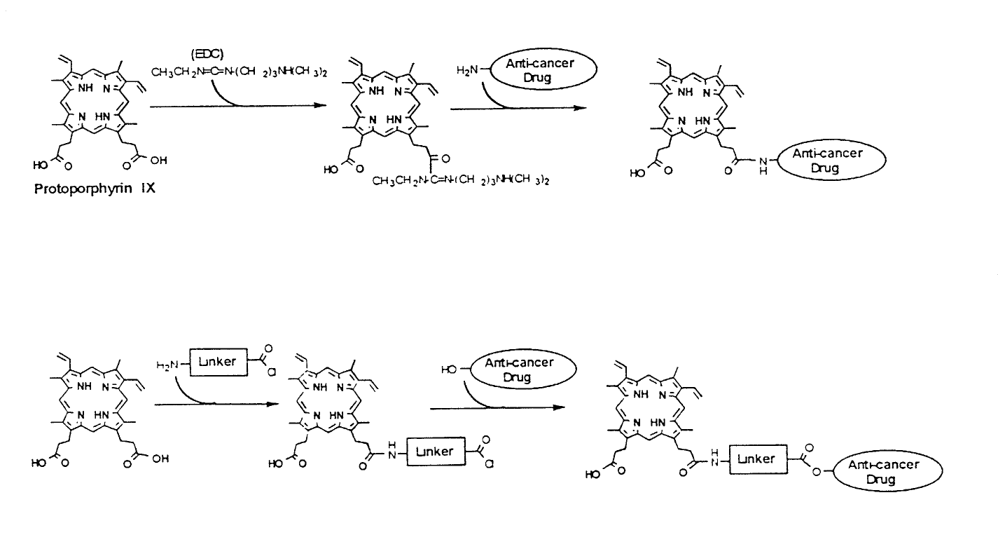

### DETAILED DESCRIPTION OF THE INVENTION

The above described drawing figures illustrate the invention, a method of targeted delivery of an anti-cancer drug and/or protease inhibitors to tumors. The invention utilizes a novel anti-cancer substance that include a porphyrin-like molecule conjugated to an anti-cancer drug. This method utilizes the inherent tendency of many porphyrins-like molecules to concentrate in tumors. In one embodiment, the substance is taken directly into one of the cancer cells of the tumor; and, once inside the cell, the anti-cancer drug acts to destroy the cell, either by cross-linking the cell's DNA or other mechanism. In this first embodiment, it is not necessary to cleave the bond between the porphyrin-like molecule and the anti-cancer drug. In a second embodiment, the substance uses a peptide chain to connect the porphyrin-like molecule to the anti-cancer drug. This embodiment takes further advantage of the high level of protease activity in tumors. The porphyrin-like molecule cannot be taken into a cell while the peptide chain is intact due to its size. However, the peptide chain is designed to be cleaved under physiological conditions surrounding the tumor. In the preferred embodiment, the peptide chain functions as a protease inhibitor once it has been cleaved.

The anti-cancer substance and its method of use have two major benefits. The first benefit is that the substance is highly selective to cancer cells. This selectivity is based upon (i) the porphyrin-like molecules' tendency to concentrate within tumors, (ii) the high metabolic rate of cancer cells (with respect to the first embodiment), and (iii) the activation of the substance in response to cleavage of the peptide chain by protease activity concentrated around the tumor (in the second embodiment). The second benefit is that the porphyrin-like molecules can also simultaneously be used in chemotherapy and/or a radiation therapy as a photosensitizer, as described in the prior art. Tile use of porphyrin-like molecules conjugated with an anti-cancer drug for targeted delivery of cancer drugs and protease inhibitors to tumors may significantly prevent and eradicate primary and secondary tumors.

Porphyrin-like Molecules

For purposes of this application, we will refer to “porphyrin-like molecules” to refer to a class of molecules and their derivatives including but not limited to the following: porphin, porphyrin, corrin, chlorin, and derivatives of these molecules, including but not limited to the following: benzoporphyrin, texaphyrin, tetrabenztriazaporphyrin, azaporphyrin, boronated metalloporphyrine, hydro-monobenzoporphyrin, heme, vitamin B12, chlorophyll, texaphyrn, tetra-hydro porphyrin, polyether-substituted porphyrin, boronated metalloporphyrin, 5,10,15,20-tetrakis(carboxyphenyl)porphyrin, azaporphyrin, benzoporphyrin, texaphyrin, texaphyrin derivatives, tetrabenztriazaporphyrin, hydro-monobenzoporphyrin, Etioporphrin-I, Octaethylporphyrin, Deuteroporphyrin-IX, Mesoporphyrin, Hematoporphyrin-IX, Protoporphyrin-IX, Coproporphyrin-I and -III, Uroporphyrin-I and -III, Chlorocruorporphrin, Pemptoporphyrin, Deuteroporphyrin-IX 2,4-(di-acrylic acid, 2,4-Diformyldeuteroporphyrin-IX, Deuteroporphyrin-IX 2,4-disulfonic acid, Phylloporphyrin-XV, Pyrroporphyrin-XV, Rhodoporphyrin-XV, Phylloerythyrin, Desoxophylloerythin, Pheoporphyrin-a5, and other porphyrin derivatives. These porphyrin-like molecules exhibit the preferred accumulation within tumors, where they are readily taken into the cancerous cells to feed the rapid metabolism of the cancerous cells. Many) of the porphyrin-like molecules tend to absorb X-ray energy to produce cytotoxic free radicals, as has been shown in the following U.S. patents, hereby incorporated by reference: 5,733,903, 5,707,608, 5,641,878, 5,622,685, 5,525,325, 5,498,710, 5,391,547, 5,389,378, 5,369,101, 5,308,608, 5,162,519, 5,162,231, 4,992,257, 4,783,529.

Porphyrin-like molecules are selectively localized on malignant neoplastic cells where considerable energy usage and metabolism occurs, as shown in the following U.S. patents, hereby incorporated by reference: 5,733,903, 5,622,685, 5,162,519, 5,162,231, 4,992,257, 4,783,529. For example, texaphyrins were shown to be localized at five to fifteen times higher concentration in tumors than in surrounding normal tissues in pre-clinical testing, as shown in U.S. Pat. No. 5,733,903, hereby incorporated by reference. porphyrin-like molecules are specifically localized in atheroma, leukemia, lymphoma, sarcoma, or other carcinoma, as shown in U.S. Pat. No. 5,451,576, hereby incorporated by refeernce. Many porphyrin derivatives have been synthesized and examined for tumor localization. As shown in U.S. Pat. Nos. 5,733,903, 5,622,685, 5,162,519, 5,162,231, 4,992,257, 4,783,529, hereby incorporated by reference, the following porphyrin-like molecules may be useful for practicing this invention: texaphyrn, tetra-hydro porphyrin, polyether-substituted porphyrin, boronated metalloporphyrin, 5,10,15,20-tetrakis(carboxyphenyl)porphyrin, azaporphyrin, benzoporphyrin, texaphyrin, texaphyrin derivatives, tetrabenztriazaporphyrin, and hydro-monobenzoporphyrin. Those skilled in the art can devise additional similar molecules with similar behavior, and minor modifications to molecules in this class should not he considered to avoid the scope of this invention.

Conjugation of A Porphyrin-like Molecule to an Anti-cancer Drug or a Peptide Chain

The porphyrin-like molecules contain, or can be modified to contain, diverse functional groups, as shown in U.S. Pat. Nos. 5,733,903, 5,707,608, 5,641,878, 5,622,685, 5,525,325, 5,498,710, 5,391,547, 5,389,378, 5,369,101, 5,308,608, 5,162,519, 5,162,231, 4,992,257, 4,783,529, hereby incorporated by reference. These functional groups can be used by those skilled in the art to conjugate the porphyrin-like molecules to either the peptide chain or the anti-cancer drug. These functional groups include but are not limited to the following: carboxyl, hydroxyl, alkyl hydroxylalkyl, oxyalkyl, oxyhydroxyalkyl, saccharide, carboxyl, carboxyamidealkyl, aromatic amino, phenolic hydroxyl, and polyethylene glycol.

The anti-cancer drug, or a peptide chain, contains or can be modified to contain one of several side chains including but not limited to the following: amines, guanidine, methyl thioether, sulfhydryl, indole, imino, imidazole, hydroxyl, phosphloryl chloride, acyl chloride, amino, thiol, imino, isocyante, acetyl, sulfate, sulfonyl chloride, phosphate, or carboxyl acid groups. Coupling reactions include but are not limited to the following: diazonium coupling, isothiocyano coupling, hydrazide coupling, amide formation, disulfide coupling, dimethylacetyl coupling, maleic anhydride coupling, thiolactone coupling, and dichlotriazine coupling. These coupling reactions between two functional groups have been well documented and are considered well known to those skilled in the art. For example, a carboxyl group in porphyrin can be covalently coupled to amino group in a peptide using coupling agents such as 1-ethyl-3-(3-(dimethylaminoprophyl) carbodiimide hydrochloride (EDC) and dicyclohexylcarbodiimde. FI)C activates carboxyl acid group which then reacts with an amino group in a peptide resulting in the formation of a covalent amide bond between the carboxyl acid group and the amino group. This has been shown in Anal Lett. 15, 147-160 1982, J. Biochem 92 1413-1424 1982.

A primary amino group in a peptide chain can also be conjugated to anti-cancer drugs such as methotrexate, daunomycin, mitomycin, vincristine, and vinca alkaloids using coupling agents and/or cross-linking agents such as benzyl carbamate, carbonate, N-succinimidyl 3-(2-pyridyldithio) propionate (SPDP), sulfo-LC-SPDP, succinimidyl 4-(N-maleimidomethyl) cyclohexane-1-carboxylate (SMCC), sulfo-SMCC, m-maleimidobenzoyl-N-hydroxysuccinimide ester (MBS), sulfo-MBS, N-succinimidyl (4-iodoacethyl) aminobenzoate (SIAB), sulfo-SIAB, succinimidyl 4-(p-maleimidophenyl) butyrate (SMPB), sulfo-SMPB, dithiobis (succinimidylpropionate), 3,3′-dithiobis (succinimidylpropionate), disuccinimidyl suberate, bis (sulfosuccinimidyl) suberate, disuccinimidyl tartarate (DST), sulfo-DST, bis[2-(succinimidooxycarbonyloxy)ethyl]sulfone (BSOCOES), sulfo-BSOCOES, ethylene glycolbis(disuccinimidylsuccinate (EGS), sulfo-EGS, etc. Anti-cancer drugs which have several pendant functional groups such as thiol, hydroxyl, acyl chloride, sulfate, sulfonyl chloride, phosphate, phosphate chloride, and imide can also be conjugated to a porphyrin-like molecule or a peptide chain using the above coupling agents and crossing-linking agents.

Cancer Cells Raise the Concentration of Various Enzymes and Growth Factors

In its preferred embodiment, the porphyrin-like molecule is conjugated to a peptide chain, and the peptide chain is conjugated to the anti-cancer drug. Tile benefit of this structure is that the peptide chain provides a cleavage site that can be customized to be cleavable under physiological conditions found in the vicinity of a tumor. In its most preferred form, the peptide chain acts as a protease inhibitor once cleaved. Those skilled in the art can devise alternative embodiments that function the same as the preferred embodiments herein disclosed without deviating from the scope of this invention. The mechanism currently preferred for use in this invention requires a peptide chain that is cleaved by a protease that is common in the vicinity of tumors.

To understand the function of the peptide chain, it is necessary to understand the behavior of typical cancer cells. Cancerous cells must produce abnormal levels of various enzymes and growth factors to support their rapid growth and metastasis. As described above, a tumor must induce angiogenesis, a process of capillary network formation, to supply nutrients inside of cancer cells. In order to form a capillary in the tumor, cancer cells secret growth factors lo such as vascular endothelial growth factors and fibroblast growth factors to induce angiogenesis from endothelial cells. Tile endothelial cells respond to the signals, and move toward the source of the signal. In the process of breaching the basal lamina that surrounds an existing blood vessel, the endothelial cells produce proteases, which enable them to digest their way through the basal lamina of the parent capillary or venule. The basal laminae is made of various proteins, including: type IV collagen, laminin, entactin, and perlecan. To digest vascular basal laminae and/or extracelluar matrix, extracelluar proteolytic enzymes are locally secreted by cancer cells. Most of these proteases are metalloproteases such as the collagenases and serine proteases such as plasmin and urokinase-type plasminogen activator (U-PA).

While U-PA and plasmin cleave a variety of proteins such as fibrin, fibronectin, and laminin with a broad specificity, collagenases cleave highly specific positions of proteins. By devising a peptide chain that contains the cleavage site of the collegenase that is prevalent in the vicinity of the tumor to be treated, the anti-cancer substance can be made specific to a particular tumor. The porphyrin-like molecule will naturally accumulate around the tumor, as described above, and the collegenases already present around the tumor will cleave the peptide chain and release the anti-cancer drug for activity.

Type IV collagen is one of the major structural protein of the basal lamina forming collagen fibrils. Type IV collagen connects the basal lamina to underlying connective tissue. The metalloprotenases such as interstitial collegenase, type IV collagenase, and stromelysin degrade components of connective tissue. Gelatinase A (72-kD) and gelatinase B (92-kD) have been reported to be a type IV collagenase. The catalytic site is nearly identical in the two collagenase types. It has been known that the 72-kd type IV collagenase secreted by cancer cells is involved in metastasis by degradation of type IV collagen of lamina, as shown in FEBS Lett. 1993; 319:35-39. The 72-kd type IV collagenase preferentially cleaves between glycine and an hydrophobic amino acid such as leucine, isoleucine, phenylalanine, valine, and alanine in collagenous (Glycine-X-Y-Glycine-X-Y-) sequences, as shown in Matrix 1993;13:181-186, J. Biol Chem 1985; 260:13601-13606, J. Natl. Cancer Inst. 1993; 85:1758-1764.

Structure of the Peptide Chain

Several research groups have synthesized specially designed peptides such as Ac-proline-leucine-glycine-S-leucine-leucine-glycine-OC₂H₅, dinitrophenyl-proline-leucine-glycine-leucine-tryptophan-alanine-arginine, and Ac-glutamate-hydroxylproline-glycine-proline-alanine-glycine-valine-arginine-glycine-glutamate-hydroxylproline-glycine that are cleaved by type IV collagenases. This work is shown in J. Natl. Cancer Inst. 1993;85:1758-1764, Biochim. Biophys. Acta 1996;1293:259-266. Type IV collagenase activities are changed by different peptide sequences, as described in Biochim. Biophys. Acta 1996;1293:259-266.

In its preferred embodiment, the peptide chain includes a sequence having the formula:

“gylcine-aa₁-aa₂-glycine”, wherein aa₁ and aa₂ are hydrophobic amino acids. This structure is targeted by the type IV collagenases, as described above. The type IV collagenases cleave the peptide chain after the first glycine. In its most preferred embodiment, the peptide chain has the formula aa₁-aa₂-aa₃-aa₄-aa₅-aa₆-aa₇-aa₈-aa₉-aa₁₀-aa₁₁-aa₁₂, wherein: aa₁ is an amino acid selected from the group consisting of arginine, lysine, tyrosine, serine, or histidine; aa₂ is an amino acid selected from the group consisting of arginine glycine, or proline; aa₃ and aa₄ are an acid amino acid selected from the group consisting of aspartate or glutamate; aa₅ is an amino acid selected from the group consisting of glycine; aa₆ and aa₇ are an amino acid selected from the group consisting of proline, leucine, isoleucine, or valine; aa₈ is an amino acid selected from the group consisting of glycine; aa₉ is an amino acid selected from the group consisting of leucine, valine, and isoleucine; aa₁₀ is an hydrophobic amino acid selected from the group consisting of phenylalanine, or tryptophane; aa₁₁ is an amino acid selected from the group consisting of alanine, valine, leucine, and isoleucine; and aa₁₂ is an amino acid selected from the group consisting of cysteine, lysine, arginine, serine, histidine, tyrosine, aspartate and glutamate. Not only does this sequence include the type IV collagenase cleaving site, it also includes a protease inhibitor. Once this sequence has been cleaved as described above, the fragment including aa₈ through aa₁₂ functions as a protease inhibitor.

Another feature worth noting is the function of arginine and tysine, if used in the selected peptide chain. Both of these amino acids can be switched from “cleavable” to “non-cleavable” by controlling the sterio-configuration of the amino acid used (L-configurations are cleavable, while D-configurations are not cleavable). This gives the user even more control over the activity of the substance in vivo. Furthermore, if cycteine is used in aa₁₂ it provides a free sulfhydryl that is available for conjugation. If another amino acid is used, aa₁₂ can be modified to include an appropriate functional group such as acyl chloride, acetyl, thioester, enolate, or any other functional group as described above.

Method of Treatment

The invention includes a method of treatment of a tumor using the above described substance. A porphyrin-like molecule is provided that exhibits preferred accumulation in the tumor. The porphyrin-like molecule having a porphyrin functional group, as described above. A peptide chain that is cleavable under physiological conditions surrounding the tumor is provided. The peptide chain has a first end and a second end; the first end has a first peptide functional group; and the second end having a second peptide functional group. The first peptide functional group is then allowed to react with the porphyrin functional group to conjugate the porphyrin-like molecule to the peptide chain. An anti-cancer drug having a drug functional group is then provided, and the drug functional group is allowed to react with the second peptide functional group to conjugate the anti-cancer drug to the peptide chain. The resulting anti-cancer substance is then administered to a patient in a pharmaceutically acceptable carrier. This process can be performed in conjunction with traditional radiation therapy. The porphyrin-like molecules retain their usefulness as photosensitizers, functioning to absorb X-ray energy to produce cytotoxic free radicals.

While the invention has been described with reference to at least one preferred embodiment, it is to be clearly understood by those skilled in the art that the invention is not limited thereto. Rather, the scope of the invention is to be interpreted only in conjunction with the appended claims.

What is claimed is:

1. An anti-cancer substance which exhibits preferred accumulation in the tumor, the substance comprising: a porphyrin-like molecule conjugated to an anti-cancer drug.

2. The substance of claim 1 wherein the porphyrin-like molecule is selected from the group consisting of: porphyrin, heme, vitamin B12, chlorophyll, texaphyrn, tetra-hydro porphyrin, polyether-substituted porphyrin, boronated metalloporphyrin, 5,10,15,20-tetrakis(carboxyphenyl)porphyrin, azaporphyrin, benzoporphyrin, texaphyrin, texaphyrin derivatives, tetrabenztriazaporphyrin, hydro-monobenzoporphyrin, Etioporphrin-I, Octaethylporphyrin, Deuteroporphyrin-IX, Mesoporphyrin, Hematoporphyrin-IX, Protoporphyrin-IX, Coproporphyrin-I and -III, Uroporphyrin-I and -III, Chlorocruorporphrin, Pemptoporphyrin, Deuteroporphyrin-IX 2,4-di-acrylic acid, 2,4-Diformyldeuteroporphyrin-IX, Deuteroporphyrin-IX 2,4-disulfonic acid, Phylloporphyrin-XV, Pyrroporphyrin-XV, Rhodoporphyrin-XV, Phylloerythyrin, Desoxophylloerythin, and Pheoporphyrin-a5.

3. The substance of claim 1 wherein the anti-cancer drug is selected from the group consisting of: methotrexate, 6-mercaptopurine, 6-thioguanine, 5-fluorouracil, cytarabine, dactinomycin, doxorubicin, daunorubicin, bleomycin, plicamycin, mechlorethamine, cyclophosphamide, carmustine, iomustine, vincristine, vinblastine, taxol, prednisone, tamoxifen, estrogens, leuprolide, interferon, cisplatin, procarbazine, asparaginase, etoposide, minocycline, bis-phosphonates, recin, metalloproteinase inhibitors, serine protenase inhibitors, and angiogenesis inhibitors.

4. The substance of claim 1 wherein the porphyrin-like molecule is directly conjugated to the anti-cancer drug using a coupling agent.

5. The substance of claim 1 wherein the porphyrin-like molecule is cross-linked to the anti-cancer drug with a cross linking agent.

6. The substance of claim 5 wherein the covalent bond can be cleaved by proteases, hydrolysis, or free radicals which are produced when the porphyrin-like molecule is exposed to X-ray energy.

7. The substance of claim 5 wherein the covalent bond is formed by a coupling reaction selected from the group consisting of the following: diazonium coupling, isothiocyano coupling, hydrazide coupling, amide formation, disulfide coupling, dimethylacetyl coupling, maleic anhydride coupling, thiolactone coupling, and dichlotriazine coupling.

8. An anti-cancer substance which exhibits preferred accumulation in the tumor, the substance comprising:
a porphyrin-like molecule conjugated to a peptide chain, the peptide chain being conjugated to an anti-cancer drug, the peptide chain being cleavable under the physiological conditions surrounding the tumor.

9. The substance of claim 8 wherein the porphyrin-like molecule is selected from the group consisting of: porphyrin, heme, vitamin B12, chlorophyll, texaphyrn, tetra-hydro porphyrin, polyether-substituted porphyrin, boronated metalloporphyrin, 5,10,15,20-tetrakis(carboxyphenyl)porphyrin, azaporphyrin, benzoporphyrin, texaphyrin, texaphyrin derivatives, tetrabenztriazaporphyrin, hydro-monobenzoporphyrin, Etioporphrin-I, Octaethylporphyrin, Deuteroporphyrin-IX, Mesoporphyrin, Hematoporphyrin-IX, Protoporphyrin-IX, Coproporphyrin-I and -III, Uroporphyrin-I and -III, Chlorocruorporphrin, Pemptoporphyrin, Deuteroporphyrin-IX 2,4-di-acrylic acid, 2,4-Diformyldeuteroporphyrin-IX, Deuteroporphyrin-IX 2,4-disulfonic acid, Phylloporphyrin-XV, Pyrroporphyrin-XV, Rhodoporphyrin-XV, Phylloerythyrin, Desoxophylloerythin, and Pheoporphyrin-a5.

10. The substance of claim 8 wherein the anti-cancer drug is selected from the group consisting of: methotrexate, 6-mercaptopurine, 6-thioguanine, 5-fluorouracil, cytarabine, dactinomycin, doxorubicin, daunorubicin, bleomycin, plicamycin, mechlorethamine, cyclophosphamide, carmustine, iomustine, vincristine, vinblastine, taxol, prednisone, tamoxifen, estrogens, leuprolide, interferon, cisplatin, procarbazine, asparaginase, etoposide, minocycline, bis-phosphonates, recin, metalloproteinase inhibitors, serine protenase inhibitors, and angiogenesis inhibitors.

11. The substance of claim 8 wherein the porphyrin-like molecule is directly conjugated to the peptide chain, which is coupled to the anti-cancer drug using a coupling agent.

12. The substance of claim 8 wherein the porphyrin-like molecule is cross linked to the peptide chain, which is cross linked to the anti-cancer drug using a cross linking agent.

13. The substance of claim 12 wherein the covalent bond can be cleaved by a mechanism selected from the group consisting of: a protease, hydrolysis, and free radicals which are produced when the porphyrin-like molecule is exposed to X-ray energy.

14. The substance of claim 12 wherein the covalent bond is formed by a coupling reaction selected from the group consisting of the following: diazonium coupling, isothiocyano coupling, hydrazide coupling, amide formation, disulfide coupling, dimethylacetyl coupling, maleic anhydride coupling, thiolactone coupling, and dichlotriazine coupling.

15. The substance of claim 8 wherein the peptide chain includes a sequence having the formula aa₁-aa₂-aa₃-aa₄, wherein:
aa₁ is the amino acid glycine;
aa₂ and aa₃ are hydrophobic amino acids;
aa₄ is the amino acid glycine.

16. The substance of claim 8 wherein the peptide chain includes a sequence having the formula aa₁-aa₂-aa₃-aa₄-aa₅-aa₆-aa₇-aa₈-aa₉-aa₁₀-aa₁₁-aa₁₂, wherein:
aa₁ is an amino acid selected from the group consisting of arginine, lysine, tyrosine, serine, and histidine;
aa₂ is an amino acid selected from the group consisting of arginine glycine, and proline;
aa₃ and aa₄ are an acid amino acid selected from the group consisting of aspartate and glutamate;
aa₅ is glycine;
aa₆ and aa₇ are an amino acid selected from the group consisting of proline, leucine, isoleucine, and valine;
aa₈ is glycine;
aa₉ is an amino acid selected from the group consisting of leucine, valine, and isoleucine;
aa₁₀ is an hydrophobic amino acid selected from the group consisting of phenylalanine, and tryptophane;
aa₁₁ is an amino acid selected from the group consisting of alanine, valine, leucine, and isoleucine; and
aa₁₂ is an amino acid selected from the group consisting of cysteine, lysine, arginine, serine, histidine, tyrosine, aspartate and glutamate.

17. A method of treatment of a tumor, the method comprising the steps of:
a) providing a porphyrin-like molecule that exhibits preferred accumulation in the tumor, the porphyrin-like molecule having a porphyrin functional group;
b) providing a peptide chain that is cleavable under physiological conditions surrounding the tumor, the peptide chain having a first end and a second end, the first end having a first peptide functional group and the second end having a second peptide functional group;
c) reacting the first peptide functional group with the porphyrin functional group to conjugate the porphyrin-like molecule to the peptide chain;
d) providing an anti-cancer drug having a drug functional group;
e) reacting the drug functional group with the second peptide functional group to conjugate the anti-cancer drug to the peptide chain; and
f) administering the anti-cancer substance in a pharmaceutically acceptable carrier.

18. The method of claim 17 wherein the porphyrin-like molecule is selected from the group consisting of: porphyrin, heme, vitamin B12, chlorophyll, texaphyrn, tetra-hydro porphyrin, polyether-substituted porphyrin, boronated metalloporphyrin, 5,10,15,20-tetrakis(carboxyphenyl)porphyrin, azaporphyrin, benzoporphyrin, texaphyrin, texaphyrin derivatives, tetrabenztriazaporphyrin, hydro-monobenzoporphyrin, Etioporphrin-I, Octaethylporphyrin, Deuteroporphyrin-IX, Mesoporphyrin, Hematoporphyrin-IX, Protoporphyrin-IX, Coproporphyrin-I and -III, Uroporphyrin-I and -III, Chlorocruorporphrin, Pemptoporphyrin, Deuteroporphyrin-IX 2,4-di-acrylic acid, 2,4-Diformyldeuteroporphyrin-IX, Deuteroporphyrin-IX 2,4-disulfonic acid, Phylloporphyrin-XV, Pyrroporphyrin-XV, Rhodoporphyrin-XV, Phiylloerythyrin, Desoxophylloerythin, and Pheoporphyrin-a5.

19. The method of claim 17 wherein the peptide chain includes a sequence having the formula aa₁-aa₂-aa₃-aa₄, wherein:
aa₁ is the amino acid glycine;
aa₂ and aa₃ are hydrophobic amino acids; and
aa₄ is the amino acid glycine.

20. The method of claim 17 wherein the peptide chain includes a sequence having tile formula aa₁-aa₂-aa₃-aa₄-aa₅-aa₆-aa₇-aa₈-aa₉-aa₁₀-aa₁₁-aa₁₂, wherein:
aa₁ is an amino acid selected from the group consisting of arginine , lysine, tyrosine, serine, and histidine;
aa₂ is an amino acid selected from the group consisting of arginine glycine, and proline;
aa₃ and aa₄ are an acid amino acid selected from the group consisting of aspartate and glutamate;
aa₅ is glycine;
aa₆ and aa₇ are an amino acid selected from the group consisting of proline, leucine, isoleucine, and valine;
aa₈ is glycine;
aa₉ is an amino acid selected from the group consisting of leucine, valine, and isoleucine;
aa₁₀ is an hydrophobic amino acid selected from the group consisting of phenylalanine, and tryptophane;
aa₁₁ is an amino acid selected from the group consisting of alanine, valine, leucine, and isoleucine; and
aa₁₂ is an amino acid selected from the group consisting of cysteine, lysine, arginine, serine, histidine, tyrosine, aspartate and glutamate.
